(19) United States
(12) Patent Application Publication
(10) Pub. No.: US 2019/0357761 A1
(43) Pub. Date: Nov. 28, 2019

(54) PROTECTIVE CAP FOR AN IMAGING DEVICE

(71) Applicant: Westfaelische Wilhelms-Universitaet Muenster, Münster (DE)

(72) Inventors: Michael Hessler, Münster (DE); Philip-Helge Arnemann, Münster (DE); Christian Ertmer, Münster (DE)

(21) Appl. No.: 16/461,758

(22) PCT Filed: Nov. 17, 2017
(86) PCT No.: PCT/EP2017/079569

§ 371 Date: May 16, 2019

(30) Foreign Application Priority Data
Nov. 17, 2016 (DE) 10 2016 122 102.3

(51) Int. Cl. A61B 1/24; A61B 1/00; A61B 1/07; A61B 1/06; A61B 5/1455

(52) U.S. Cl. CPC A61B 1/24; A61B 1/00137; A61B 5/1455; A61B 1/0646; A61B 1/07

(57) ABSTRACT

Disclosed is a protective cap (400) for an imaging device, the cap having a hollow protective cap body with a front end (404), a cylindrical surface (402) and an open rear end (406), through which the imaging device can be inserted into the protective cap, and a depression (408) provided in the front end. The protective cap also has at least one channel (502) provided at the front part of the protective cap body, beneath the front end, the at least one channel having a first opening that is provided in the lateral wall (412) of the depression and a second opening that is provided preferably in the cylindrical surface of the protective cap body.

The present device is a protective cap for an imaging device, in particular a protective cap for a camera for monitoring a microcirculation, for instance in the mouth of a patient.

The life-threatening diseases of intensive care patients are often accompanied by changes in microcirculation (also called microvascular perfusion). Microcirculation is increasingly regarded as a critical determinant of the organ function of medical conditions requiring intensive care. For that reason, in recent years, new methods have been developed for the visualization of microcirculation, such as sidestream dark field imaging (SDF for short) and incident dark field illumination imaging. By means of these methods, a direct observation of the microcirculation at the bedside of the patient can be performed based on appropriate examination devices. The study of microcirculation is currently mainly conducted in the context of university medicine. The direct examination of microcirculation is a valuable diagnostic tool and can contribute to an improved assessment of the stage of disease in an ICU patient. It can be assumed that in the upcoming years therapy decisions will be made on the basis of microcirculatory parameters.

As a rule, as part of the methods listed above, the microcirculation of patients is examined by inserting a small, pin-shaped camera into the mouth, placing the camera tip under the tongue (sublingually). The camera is covered by a transparent low-germ protective cap, which for hygienic reasons is removed from the camera and disposed of after a patient has been examined. To examine the next patient, a new protective cap is placed on the camera. In this context, CytoCam® by Braedius is mentioned here by way of example. It is a camera for monitoring the microcirculation by a company called Braedius, which camera is based on incident light dark field imaging and is ideally suited to a direct visualization of the sublingual microcirculation. The disposable protective caps described are used for this camera as well.

In general, sublingual microcirculation is monitored by placing a camera equipped with a protective cap on or in contact with the sublingual mucosa. In this way, the capillaries below the mucosal surface can be visualized.

A major problem in the study of microcirculation is the low intraluminal pressure in the capillaries. When the camera is placed on the sublingual mucosa, the pressure on the sublingual mucosa caused by inserting the camera is sufficient to block the capillaries. This may sometimes suggest a poor perfusion of the microcirculation, although there actually is no disruption of microvascular perfusion. This circumstance is also called pressure artifact and may ultimately result in a misdiagnosis of the health status of an intensive care patient and thus, most likely, in adverse treatment decisions for the patient.

A further problem in the study of microcirculation is the presence of excessive saliva, cell debris (e.g. red blood cells) or air bubbles on the floor of the mouth. The presence of these materials in the examination area considerably complicates testing microcirculation and may even render the testing of microcirculation impossible.

The present invention addresses the problem of how to circumvent the above-mentioned problems in testing microcirculation. These problems are solved by the device according to claim 1. Further preferred embodiments are described in the dependent claims.

Before the invention is explained in more detail, first a protective cap for microcirculation imaging devices known from the prior art will be briefly described. An example of such a protective cap is shown schematically in FIG. 1 and will be described in detail below. The protective caps of the microcirculation observation cameras currently available are made of a transparent plastic, through which the microcirculation can be observed by means of the camera. Due to the pin-shaped form of the camera, generic protective caps are also substantially pin-shaped or conical, to enable the pin-shaped part of the camera, at the end of the optics, to be inserted into the protective cap. The generic protective cap is rigid and made of a transparent plastic having a thickness of approximately 1 mm. The protective cap is placed over the rod-shaped end of the camera before the examination and closes tightly around the camera body. To observe the microcirculation, the protective cap is placed directly on the mucosa. The contact surface of the protective cap and the mucous membrane is a flat surface.

Based on this situation, the invention addresses the problem of improving the testing of microcirculation, in particular to prevent the occurrence of pressure artifacts and of further improving the imaging quality, which may be impaired by excessive saliva, cell debris and/or air bubbles.

The device according to the invention is a protective cap for an imaging device, which has a hollow cap body having a front face, a lateral surface and an open rear end, through which the imaging device can be inserted into the protective cap, and a recess, which is arranged in the front face. The protective cap also has at least one channel, which is arranged in the front part of the body of the protective cap beneath the front face, wherein the at least one channel has a first opening, which is arranged in the side wall of the recess, and a second opening, which is preferably arranged in the lateral surface of the body of the protective cap.

Deviating from hitherto known protective caps which, as mentioned above, have a continuously plane front face, the protective cap according to the invention is modified in such a way that it has a depression (recess) in the front face. Due to this modification pressure artifacts in the examination area can be reduced or even eliminated. In the case of generic protective caps, pressure is exerted on the affected mucosa at locations where the protective cap is placed on the mucous membrane. This pressure results in the compression of superficial capillary loops, which obstructs the flow in the capillaries present in the compressed area and becomes discontinuous; pressure artifacts result. In the protective cap according to various exemplary embodiments, a recess is incorporated in the front face of the body of the protective cap, for instance, in the center of the area, which is in contact with the mucosa during the examination. In this way, microcirculation (e.g., microcavity video recording) can be performed in this area without pressure artifacts.

The protective cap according to the invention may have an elongated or pin-shaped form, for instance in the shape of a blunt cone, wherein the top surface corresponds to the front face and the base surface corresponds to the open end of the body of the protective cap. The overall shape of the protective cap can be adapted to the shape of the part of the imaging device, which it is to cover. The imaging device may be any camera suitable for monitoring microcirculation. For instance, it may be the CytoCam® by Braedius. The protective cap according to the invention can be made of any suitable material, such as a plastic, and can be made for instance by sintering or 3D printing.

According to further embodiments, the recess present in the protective cap may have a bottom. In other words, the recess may be a recess or a hollow closed against the cavity located in the interior of the body of the protective cap. In that case, the bottom can tightly seal the recess with respect to the inner cavity of the cap body (impermeable for fluids), such that fluids or other matter cannot enter the cavity of the cap through the recess. The internal cavity of the protective cap, in which a camera is inserted while the patient is examined, can be kept sterile in that way.

According to further exemplary embodiments of the protective cap, the bottom of the recess may comprise a transparent material, for instance a cover glass. The bottom of the recess may be an integral part of the entire protective cap. Alternatively, the bottom of the recess may not be integrally formed with the recess. The bottom of the recess may also be formed of a different material than that of the protective cap. In particular, the bottom may comprise a cover glass or other platelet of a translucent material. The material forming the bottom can be introduced, for instance, through the open end of the cavity into the latter and attached to the lower edge of the recess and/or in the back of the front face, for instance by a click mechanism provided for that purpose.

Wth regard to the bottom of the recess, it can have an optical element according to further exemplary embodiments of the protective cap. The bottom of the recess may be formed of a transparent material and can be configured as an optical element. For instance, the optical element may be an optical filter, a lens, an aperture, or any combination thereof. In general, the optical element may be any appropriate optical element, which participates in the design of the light path from the tissue of the examined patient to the camera in the protective cap. To name a concrete exemplary embodiment, the bottom of the recess may have electrical connections and consist of glass that can be darkened and in that way be set up as a variable aperture. In addition, an optical filter may be provided, which is arranged in front of or behind the aperture. In this application, the relationship between the bottom of the recess and the optical element is rather broad. That means, the optical element (or several optical elements) can be configured as a module (as modules) and can be coupled with an element that forms the physical bottom of the recess (i.e., the layer of material that completely seals the recess from the cavity). However, the optical element may also be directly integrated in the material layer that forms the bottom of the recess.

According to further exemplary embodiments of the protective cap, at least one retaining element, which is adapted for a detachable attachment of the bottom of the depression, may be arranged on the inside of the body of the protective cap in the area of the front face. The term ‘area of the front face of the protective cap’ can refer to the front face of the protective cap and the adjoining area of the protective cap, that is to say the front tip of the protective cap, for instance approximately the front fifth of the protective cap. In other words, the protective cap according to the invention can be set up such that the bottom of the depression is designed as an interchangeable module and can be inserted or replaced, for instance, through the open rear end of the protective cap. The retaining element may be arranged, for instance, as an annular groove, which is arranged in the inner wall of the cavity. In this case, the protective cap may consist of two parts, which can be screwed to each other or mated, wherein a first (e.g. upper) part of the annular groove is provided in the first (e.g. upper) part of the protective cap and a second (e.g. lower) part of the annular groove is provided in the second (e.g. lower) part of the protective cap. The bottom of the recess can be inserted between the two cap parts before they are screwed or mated.

According to further embodiments, the front face of the body of the protective cap may be formed flat around the recess. In this case, the plane of the front face can be arranged in parallel to the plane of the bottom of the recess. Additionally, the angle at which the lateral surface meets the front face at any point along the outer edge of the front face may optionally be about 90° or more. The larger this angle, the more pronounced the cone nature of the protective cap in comparison to a cylinder. In a similar manner, the angle between the front face and the wall of the recess can be about 90° or more. In other words, the front face can bend downwards by up to 90° and in that way form the wall of the depression that leads to the bottom of the depression. The transition between lateral surface and front face can be formed by a sharp edge or a rounded edge. The transition between the front face and the wall of the depression can in a similar manner be formed by a sharp edge or a rounded edge. The proposition that the front face of the body of the protective cap can be formed flat around the depression may concern the bigger part of the front face; this may however not apply to the edge area of the recess located in the front face and/or the area of the outer edge of the front face.

According to further exemplary embodiments, the front face of the body of the protective cap may be formed as a surface rising or falling radially towards the recess. I.e., in a cross-sectional side view the outer edge of the front face, where the front face merges into the lateral surface, may be lower or higher. This rising or falling property of the front face does not necessarily strictly apply to every area/part of the front face, but shall be taken as a proposition regarding the average behavior of the front face. For instance, the front face may, overall, ascend or drop step-like or in another segmented manner towards the opening of the depression (recess opening), but have a plane course in some sections (i.e. neither rising nor falling) or even have a slope in the opposite direction to the overall behavior. Pressure artifacts can be widely avoided around the actual tissue site being examined by using an embodiment of the protective cap having a front face radially sloping towards the recess and a rounded edge at the recess opening. At the same time, however, an outer edge of the front face in contact with the tissue can ensure that no scattered light can enter the recess and thus ultimately the imaging device from the outside.

According to further exemplary embodiments, the front face of the body of the protective cap may be patterned at least in one area around the recess. The area may be arbitrarily shaped, such as concentric or elliptical, and may be symmetrical or asymmetrical around the depression opening. The area can also extend over the entire front face. The patterning may be any appropriate patterning, such as a knurled or ribbed patterning. The patterning may include functional materials, i.e. materials having e.g. an improved grip in relation to the examined tissue (e.g., mini-suction bells or mini-suction cups) or have special optical (e.g., reflective) properties. The patterning may also be functional in that it supports the removal of excessive saliva, cell debris or air bubbles. For instance, according to further exemplary embodiments of the protective cap, at least one groove, which extends between the depression and the outer edge of the front face, can be arranged in the front face of the body of the protective cap. The at least one groove can be used as a transport channel, for instance, to remove the aforementioned unwanted material from the examination/observation area, which is essentially congruent to the recess opening.

According to further embodiments of the protective cap, it may have at least one channel, which extends in the front part of the body of the protective cap under the front face. The first channel may have a first opening, which opens or is arranged in the side wall of the depression or in the front face. In addition, the channel may preferably have a second opening, which is arranged in the lateral surface of the body of the protective cap. The channel can, on the one hand, assume the role of the above-mentioned groove and be used for the removal of unwanted material from the investigation/observation area. The material can enter the channel through the first opening, which can open into the wall of the recess. To actively suction the unwanted material, a line by means of which a negative pressure can be generated in the channel may be connected to the second opening of the channel. On the other hand, the at least one channel can also function as a supply line and be used, for instance, for introducing air or liquid, preferably transparent, media into the depression. In this case, the at least one channel has the function of a flushing channel. By introducing an optically active substance (e.g., immersion oil) into the recess, the achievable resolution of the image taken during a patient examination can be improved. On the other hand, the at least one channel may have at least one opening, which opens into the front face, for instance into an area of the front face around the recess. By providing a negative pressure in the at least one channel, this negative pressure at the at least one opening can ensure that tissue in contact with the opening is suctioned and thus secured. The at least one channel can therefore fulfill a variety of functions, wherein various parameters such as its dimensions, its course, the number and location of the openings can be adapted to the intended use. Overall, of course, exemplary embodiments of the protective cap having multiple channels are included in the invention, wherein any combination of the types of channels described above can occur in one and the same protective cap. For instance, under the channels extending in the front part of the protective cap, at least one channel can serve as a flushing channel and at least one further channel can serve as a supply line. In general, it may be advantageous if the opening of the channel serving as a line for the removal of undesirable material is arranged substantially opposite from the opening of the channel serving as a flushing channel, for instance arranged diametrically opposite.

When using the channel as a supply line, a viscous liquid can be introduced into the recess as the transparent liquid medium. The introduction of the liquid medium into the recess can be continuous or as a bolus, for instance by means of a syringe or pump that can be connected to the at least one channel. At least one further channel or at least one furrow mentioned above can be used to remove material from the recess, which material may be detritus and/or saliva, or the liquid medium diluted by saliva, such as the viscous gel. For this purpose, a syringe or a pump can be coupled to the outer opening of the removal channel and the extraction process can be continuous or intermittent. To minimize the risk of blockage or blocking of the discharge channel, the diameter of a discharge channel may be greater than the diameter of the channel used for the introduction of the liquid medium.

In addition, introducing a medium into the recess may be beneficial, particularly a viscous gel, because it can build up a slight counter pressure on the observed tissue (i.e., the tissue in the area of the recess). As a result, the tissue in the observation area (i.e., in the area of the depression of the protective cap) can be prevented from “falling” into the depression or expanding, and thereby blocking capillaries in the tissue at the edge area of the depression. In other words, the liquid medium introduced into the depression can ensure that the tissue arranged in the area of the depression bulges only slightly in the direction of the depression. To prevent too much counter pressure from building up due to the medium introduced into the depression, grooves or furrows and/or other channels, which extend radially outward from the recess to the shell area of the protective cap and thus allow drainage of the liquid medium, such as the viscous gel, may be arranged in the front face of the cap, as mentioned above. The dimension of the grooves and/or channels provided for depressurization may be adjusted to the liquid medium, for instance to its viscosity, thus allowing for its controlled discharge at a predetermined rate.

In addition, the introduction of the liquid medium into the recess during a subsequent examination can provide for active moistening of the examined mucosa, which is often very dry, in particular in intensive care patients. At the same time, the liquid and preferably viscous medium can form a lubricating film on the mucous membrane and thus improve the sliding properties of the protective cap on the mucous membrane, which can prevent injuries to the mucous membrane. In addition, the liquid medium in the recess can reduce or prevent reflections otherwise occurring at the boundary layer between air and the moist mucosa, which may interfere with mucosal observation through the protective cap.

The liquid viscous medium used may be, for instance, high viscosity fluids based on hyaluronates (e.g., MICROVISC® sodium hyaluronate 1%) or hydroxypropylmethyl cellulose (e.g., POLYVISC® 2%) known in ophthalmology.

According to further embodiments of the protective cap, the side wall of the recess and/or the front face of the protective cap may be made of an opaque material. The sidewall of the recess is generally defined as the wall extending largely downwards from the front face to the bottom of the recess. The provision of translucent material in said areas can ensure that a maximum of light from the examined area of the tissue reaches the lens of the camera. To prevent any interference due to scattered light from the environment of the examined tissue area from reaching the lens of the camera, however, the lateral surface can be made of an opaque material.

According to further embodiments, a fiber optic cable can be arranged in the at least one channel. The at least one channel can thus be used for the distribution of light, wherein the light from an external light source can be injected into the fiber optic cable and can be routed to the desired location by means of the fiber optic cable. Appropriately positioned openings in the fiber optic cable can be used to route light to desired locations of the protective cap to additionally illuminate the examined tissue. More preferably, light of one wavelength (or multiple wavelengths) may be introduced into the observed/examined tissue, which is different from the wavelength used by the camera. This can be particularly useful if only selected structures in the observed tissue are to be made visible by means of resonant excitation. To specifically visualize the microcirculation, which can be attributed to the flow of blood and thus hemoglobin, green light having a wavelength of 525 nm can be irradiated into the observed tissue.

Further advantages and features of the invention will become apparent from the following exemplary explanations with reference to the figures. The features shown in the figures and/or explained below may, regardless of specific feature combinations, be general features of the invention, which can be transferred to other exemplary embodiments of the invention transferable independent of the representations here.

FIG. 1 shows an ordinary protective cap for a camera for examining the microcirculation.

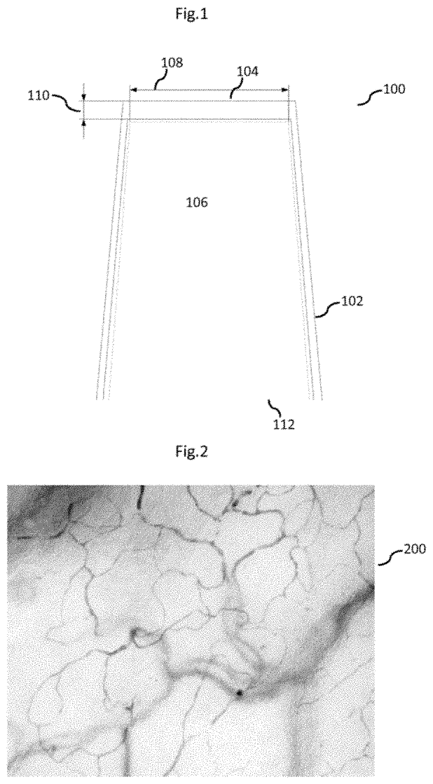

FIG. 2 shows a snapshot of an image of the sublingual microcirculation.

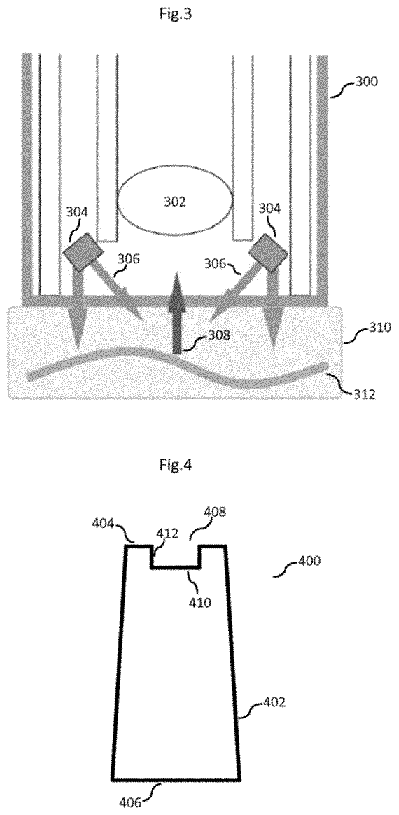

FIG. 3 shows an option for visualizing the microcirculation.

FIG. 4 shows a schematic side view of a basic form of the protective cap according to the invention.

FIG. 5 shows a further exemplary embodiment of the protective cap according to the invention.

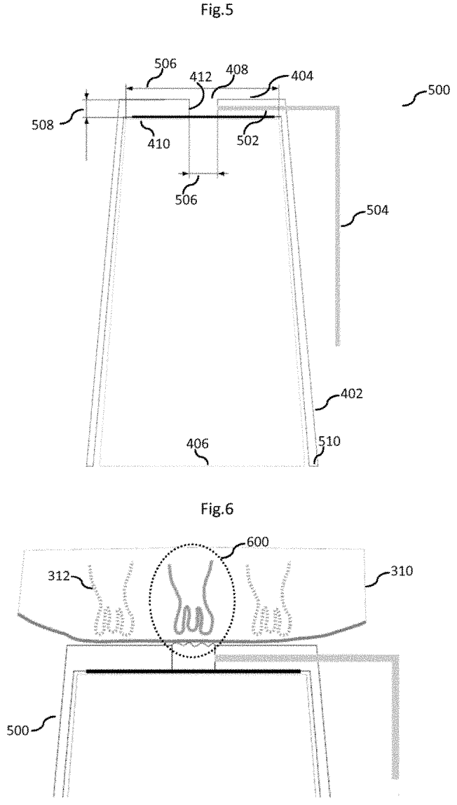

FIG. 6 illustrates the advantageous effect of the depression of the protective cap according to the invention in the examination of the microcirculation.

FIG. 7 shows a further embodiment of the protective cap.

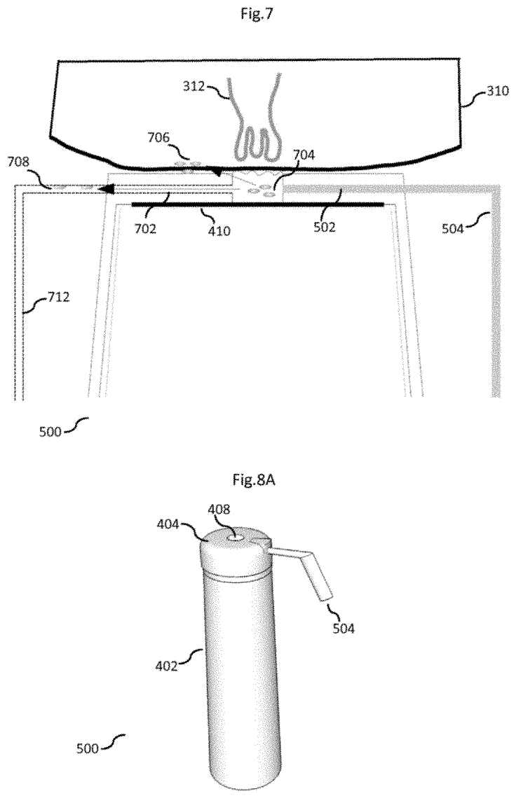

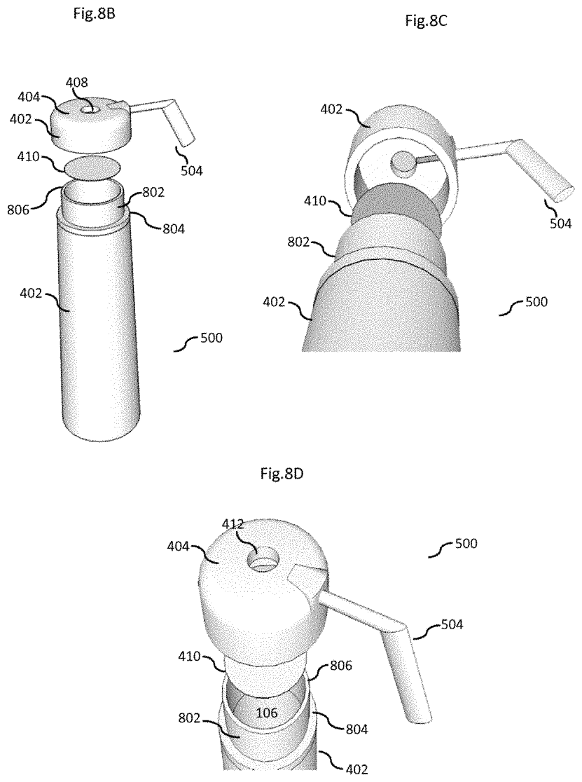

FIGS. 8A to 8D show 3D representations of the protective cap according to further exemplary embodiments.

In the figures described below, identical elements appearing in different figures bear the same reference numerals.

FIG. 1 shows an ordinary protective cap 100 for a camera for examining the microcirculation. The camera, for instance the CytoCam® by Braedius, is inserted into the hollow interior 106 of the protective cap 100 and can thus be kept sterile during an examination of the microcirculation in the oral cavity of a patient. The standard protective cap 100 is made of a plastic and is conically shaped. The rear end 112 of the protective cap, through which the camera can be inserted into the interior 106 of the protective cap, is open. A front side 104 adjoins the lateral surface 102, which front side is made of a transparent material, such that the microcirculation can be observed through the front side 104 by means of the camera. The front side 104 may be round in plan view and substantially have a diameter 108 of about 9 mm. The thickness 110 of the front 104 forming layer is about 1 mm.

FIG. 2 is a snapshot 200, which shows an image of the sublingual microcirculation, as can be recorded using a camera set up for that purpose (including a protective cap attached thereto). The image was generated by illuminating the tissue using green light at 525 nm, which is almost completely absorbed by the hemoglobin. For that reason, blood-perfused structures appear black in this illustration.

Microcirculation can be visualized, for instance, by means of incident-light dark field imaging, which will be explained below with reference to FIG. 3 (Figure from van Eiteren HA; Journal of Clinical Monitoring and Computing; 2015 October; 29 (5): 543-8). For this purpose, a suitably equipped camera can be used, which is represented in FIG. 3 by a lens 302. As shown, the pen-shaped camera is also surrounded by a protective cap 300. The protective cap 300 may be the protective cap shown in FIG. 1. The camera is equipped with additional light sources 304, e.g. with green LEDs illuminating the tissue 310, with which the protective cap 300 is brought into contact. When using green light, hemoglobin appears black because it has a broad absorption spectrum around 525 nm. As explained above, pressure artifacts are a significant problem in the assessment of the microcirculation. By placing the protective cap 300 on the tissue 310 to be examined, the fine capillaries 312 in the surface can be blocked, resulting in a distorted representation of the microcirculation and ultimately in a wrong diagnosis.

FIG. 4 shows a schematic side view of a basic form of the protective cap 400 according to the invention. Like the conventional protective cap 100 known from the prior art and illustrated in FIG. 1, the protective cap 400 according to various embodiments also has an elongate shape, which may correspond, for instance, to a cylinder or truncated cone. The body of the protective cap essentially has a lateral surface 402 and a front face 404 and an open rear end 406. In contrast to the known ordinary protective cap, however, the protective cap 400 according to the invention has a depression 408 in the front face 404. The recess 408 can be present, for instance, centered in the (viewed in plan view) circular front face 404. The recess 408 has a side wall or a wall 412, which extends to the bottom 410 of the recess 408. The geometric configuration of the front face 404 including the recess 408 can be varied. In the illustrated exemplary representation in FIG. 4, the corners at the transition of the lateral surface 402 to the front face 404 and are pointed and angular at the transition of the front face 404 to the wall 412 of the depression 408. However, any of these transitions may be rounded (regardless of the other transition). Furthermore, the angles at the transitions between the mentioned surfaces, which in FIG. 4 are approximately at 90° in the first approximation, can be set to different values. In particular, the front face 404 does not have to be flat, but may have a patterning or may rise or fall toward the edge of the recess 408. Depending on the embodiment of the protective cap 400, at least a part of the bottom 410 and optionally at least a part of the wall 412 and at least a part of the front face 404 may be made of a transparent material. This ensures that the camera located inside the protective cap 400 can map the observed tissue and also that additional light from the protective cap 400 can be radiated into the tissue if required. Finally, the ratio between the size of the opening of the recess 408 and the size of the front face 404 can also be varied according to requirements. It has to be emphasized that the cross-sectional view shown in FIG. 4 is solely a schematic representation of the protective cap according to the invention and the relations of the individual components to one another can be varied in many ways. However, all embodiments have the common feature that a recess 408 is arranged in the front face 404, preventing part of the tissue observed by the camera during an examination of the microcirculation from coming into in contact with the protective cap 400, but is pressureless because of the recess 408.

A further protective cap 500 according to various embodiments is shown in FIG. 5. Elements that have already been explained with reference to FIG. 4 will not be described again. In the exemplary embodiment of the protective cap 500 shown in FIG. 5, the bottom 410 of the recess is designed as a platelet of transparent material, which abuts the rear side of the front face 404. The material platelet may be permanently attached to the back of the front face or be detachably (in the sense of interchangeable) attached by means of appropriate retaining mechanisms. The bottom plate 410 can be replaced through the open rear end 406 of the protective cap 500. In the schematic cross-sectional view shown, dimensions are also shown to illustrate a concrete example of use, wherein the thickness 508 of the front face 404 and the diameter 506 of the front face 404 can have the same dimensions as the corresponding dimensions in conventional protective caps, in particular because of the compatibility with the commercially available cameras, i.e. approx. 1 mm and approx. 9 mm. The diameter 506 of the bottom of the recess 408 may be, for instance, 3 mm. It should be understood that these parameters may be adjusted according to requirements without departing from the spirit of the invention.

A further element is shown in the illustration of FIG. 5, which can be provided independently of all other variable properties of the cap according to the invention, namely a channel 502. Although only one channel 502 is shown, it is representative of a number of channels, which may be disposed in the material layer under the front face 404. The channel 502 has a first opening, which opens into the wall 412 of the recess 408. The channel 502 also has a second opening, which is arranged in the lateral surface 402. In the embodiment shown, a line 504 is also connected to the second opening of the channel 502. As described above, the (at least one) channel 502 may be used to create a vacuum in the recess 408, to introduce a fluid into the recess 408, or to extract unwanted material from the recess 408. The openings of a channel need not be in a 1:1 relationship with channel 502. I.e., one channel may, for instance, branch out, and the opening in the lateral surface 402 may be in contact with a plurality of openings in the wall 412 of the recess 408. Likewise, if necessary, the channel 502 may have (additionally or exclusively) at least one opening, which is arranged in the front face 404. This can be advantageous, for instance, if the tissue arranged around the depression 408 is to be suctioned and/or secured during an examination by means of underpressure. Finally, the position of the second opening in the lateral surface 402 is arbitrary. Thus, deviating from the position shown in FIG. 5, it can be arranged further towards the open rear end 406 of the protective cap 500. However, the second opening can also be arranged in the edge 510 of the lateral surface 402, wherein the channel 502 can then be integrated into the shell of the protective cap 500. Such an arrangement may have the advantage that the connected line 502 can be shorter, its detachment from the opening is less likely and that the corresponding protective cap without the laterally projecting line 504 will be more compact and thus able to penetrate into small gaps and corners of the examined tissue.

The effect of the depression of the protective cap according to the invention in the examination of the microcirculation in the tissue is illustrated with reference to FIG. 6. FIG. 1 schematically shows a tissue layer 310 in conjunction with capillaries 312 or capillary loops present therein and a protective cap 500 in contact with this tissue layer 310, in which a camera is located (not explicitly shown in FIG. 6). The illustration shows that the area of the tissue 310, on which the front face 404 of the protective cap 500 rests, is compressed and thus the blood flow through the corresponding capillaries 312 can be impeded (becomes discontinuous). In the observed area 600 of the tissue 310, over which the depression 408 is located, no such compression occurs, and thus the natural state of the microcirculation in the capillaries 312 located in this area can be observed. The flow in the capillaries 312 in the observed area 600 is not affected and thus continuous.

In general, in all embodiments of the protective cap, the space of the recess 408 can be filled, for instance, by an optically transparent, liquid medium or air (by means of the channel 502). As a result, the remaining pressure exerted on the tissue 310 is lower than that due to the contact of the front face 404 of the protective cap. Due to the option of a variable filling of the recess 408 using any media, the examination of the microcirculation under the influence of a variable pressure disruption is also conceivable, permitting inferences on the health of the patient. For instance, the recess 408 can be pressurized by supplying air at a predefined pressure, and after reducing this predefined pressure, the time, which has to pass until the microcirculation returns to its original state, can be measured. For performing these and other types of dynamic measurements, this protective cap is best suited because it provides an interface between the tissue 310 and the protective cap, on which on the one hand a pressureless area (in the sense of compression of the tissue 312) is provided and on the other hand a predefined pressure for examination purposes can be applied to the tissue 312 by targeted introduction of fluids (e.g. immersion oil) or air.

FIG. 7 shows another embodiment of the protective cap 500, wherein only its front part is shown. In this embodiment the protective cap 500 has two channels, a first channel 502 and a second channel 702, wherein both channels can be similar and are interchangeable regarding their function. In all other respects, the protective cap shown in FIG. 7 may, for instance, correspond to the protective cap shown in FIG. 5.

As previously mentioned, undesirable material 704, such as detritus (including red blood cell debris), saliva, or small air bubbles, often obstructs microcirculation vision. To eliminate these visual obstructions, the recess 408 can be flushed by means of the first channel 502, which can be used as a flushing channel, using a suitable liquid. The unwanted material 704 may then be flushed out of the recess 408 and transported away from the viewing area of the camera along the interface between the tissue 310 and the front face 404 of the protective cap 500 (first flushing path 706). For this purpose, it may be advantageous if the front face is patterned and has corresponding grooves or the like to facilitate the removal of the undesirable material. In addition, a second (or several) channel(s) 702 may be provided, through which the introduced liquid can be transported away from the depression 408 (second flushing path 708) together with the undesired material 704. As shown in FIG. 7, a line 712 can optionally be connected to the second channel 702, via which channel the second channel 702 can be negatively pressurized to actively extract the undesired material 704 from the depression 408. Otherwise, the second channel 702 may open into the lateral surface 402 of the protective cap (i.e., without an extension line 712 to a suction pump) and serve to discharge the undesired material from the recess 408.

FIGS. 8A to 8D show a 3D representation of the protective cap according to further embodiments, wherein FIG. 8A shows a spatial representation of an assembled form of the protective cap 500, while FIGS. 8B to 8D show exploded views from different angles of view. The reference numerals are based the preceding figures, such that elements of the cap according to the invention which have been described above, will not be described again.

The embodiment shown in FIGS. 8A to 8D is characterized in that the tip of the protective cap 500, essentially a part of the protective cap 500, which has the front face 404 and an adjoining area of the lateral surface 402 (hereinafter referred to as the upper part), can be detached from the remaining part, which has the remaining part of the lateral surface 402 (hereinafter referred to as the lower part). In such an embodiment, the bottom 410 can be inserted particularly easily or an already existing bottom 410 can be replaced. As illustrated, the bottom 410 may be inserted into the upper part or placed on the edge 806 of a raised ridge 802. After mating the upper and lower parts, the bottom 410 is held in position because of the pressure of the raised web 802 thereon. The lower edge of the upper part rests on a projection 804 in the lower part. In the illustrated embodiment, there is an interference fit between the two mateable parts. However, the connection of the upper part to the lower part can just as well be realized using a screw connection.

1. A protective cap for an imaging device, comprising:
a hollow protective cap body having a front face, a lateral surface and an open rear end, through which the imaging device can be inserted into the protective cap; a recess, which is arranged in the front face;
at least one channel, which is located in the front part of the body of the protective cap under the front face;
wherein the at least one channel has a first opening, which is arranged in the side wall of the recess, and has a second opening, which is preferably arranged in the lateral surface of the body of the protective cap.

2. The protective cap according to claim 1,
wherein the recess has a bottom; and
wherein the bottom preferably seals the recess against the internal cavity of the body of the protective cap.

3. The protective cap according to claim 1 or 2,
wherein the bottom comprises a transparent material, preferably a cover glass; and optionally further wherein the bottom comprises an optical element.

4. The protective cap according to any one of the claims 1 to 3,
wherein at least one retaining element, which is adapted for a detachable attachment of the bottom of the depression, may be arranged on the inside of the body of the protective cap in the area of the front face.

5. The protective cap according to any one of the claims 1 to 4,
wherein the front face of the body of the protective cap is formed flat around the recess; or
wherein the front face of the body of the protective cap is formed as a surface rising or falling radially towards the recess.

6. The protective cap according to any one of the claims 1 to 5,
wherein the front face of the body of the protective cap is patterned in at least one area around the recess.

7. The protective cap according to any one of the claims 1 to 6,
wherein at least one groove, which extends between the depression and the outer edge of the front face, is arranged in the front face of the body of the protective cap.

8. The protective cap according to any one of the claims 1 to 7, further comprising:
at least one further channel, which extends in the front part of the body of the protective cap under the front face;
wherein the at least one further channel has at least one first opening, is arranged in the front face; and
wherein the at least one further channel preferably has a second opening, which is arranged in the lateral surface of the body of the protective cap.

9. The protective cap according to any one of the claims 1 to 8,
wherein the side wall of the recess and/or the front face of the protective cap is/are made of an opaque material.

10. The protective cap according to claim 9,
wherein a fiber optic cable is arranged in the at least one channel.
